(19) United States
(12) Patent Application Publication
(10) Pub. No.: US 2025/0012782 A1
(43) Pub. Date: Jan. 9, 2025

(54) METHOD FOR SCREENING AN ACTIVE COMPOUND IN VITRO

(71) Applicant: L'OREAL, Paris (FR)

(72) Inventors: Rodrigo De Vecchi, Rio De Janeiro-RJ (BR); Lionel Breton, Aulnay-Sous-Bois (FR); Charbel Bouez, Clark, NJ (US); Vanja Dakic, Rio De Janeiro-RJ (BR)

(21) Appl. No.: 18/896,655

(22) Filed: Sep. 25, 2024

Related U.S. Application Data
(62) Division of application No. 17/255,614, filed on Dec. 23, 2020.

(51) Int. Cl. G01N 33/50; C12N 5/00; C12N 5/071; C12N 5/0793

(52) U.S. Cl. CPC G01N 33/5058; C12N 5/0075; C12N 5/0619; C12N 5/0629; C12N 5/0697; C12N 2500/44; C12N 2501/15; C12N 2501/155; C12N 2502/081

(57) ABSTRACT

The present invention relates to a kit comprising a co-culture microdevice containing peripheral sensory neurons (PSN) and human epidermal keratinocytes (HEK) in a cell culture adapted for both cell types. It is also described a method for screening an active compound using the kit according to the present invention, as well as the use thereof for in vitro drug tests and for producing a cosmetic product for various dermatological applications, such as atopic dermatitis, sensitive skin, photoaging, wound healing and epidermal thickness in aged skin.

### FIELD OF THE INVENTION

The present invention relates to a kit comprising a co-culture microdevice containing peripheral sensory neurons (PSN) and human epidermal keratinocytes (HEK) in a cell culture adapted for both cell types. It is also described a method for screening an active compound in vitro using the kit according to the present invention, as well as the use thereof for in vitro drug tests and for producing a cosmetic product for various dermatological applications, such as atopic dermatitis, sensitive skin, photoaging, wound healing and epidermal thickness in aged skin.

### BACKGROUND OF THE INVENTION

Keratinocytes and peripheral sensory neurons (PSN) have an extensive interplay during development and within the mature skin. For instance, keratinocytes release neurotrophic factors that induce arborization of free nerve endings and neurite outgrowth toward the skin surface (Albers & Davis, The skin as a neurotrophic organ. Neuroscientist. 2007; 13:371-82). They also release inflammatory mediators involved in the response to tissue damage and hypersensitivity reactions, as well as in the response to cold and heat through receptors of the TRP family of cation channels (Chung et al., TRPV3 and TRPV4 mediate warmth-evoked currents in primary mouse keratinocytes. J Biol Chem. 2004; 279:21569-75).

On the other hand, sensory endings do not only transduce sensory signals, but have an important role in the cutaneous metabolism and homeostasis through the secretion of pro-inflammatory neuropeptides and inflammatory mediators that control vascularization and tissue renewal (Roosterman et al., Neuronal control of skin function: the skin as a neuroimmunoendocrine organ. Physiol Rev. 2006; 86:1309-79). Particularly, TRPV1 positive nociceptors also regulate skin longevity and metabolism, as well as the immune response over aging (Riera et al., TRPV1 pain receptors regulate longevity and metabolism by neuropeptide signaling. Cell 2014; 157, 1023-1036).

The neuropeptides produced by the sensory neurons innervating the skin modulate cellular proliferation, wound healing, pigmentation and keratinocyte innate immune response. It is known that the neuropeptides are able to stimulate inflammatory mediators produced by keratinocytes, but there is still little information regarding the mechanism(s) thereby the neuropeptide activation of keratinocytes cell surface receptors ultimately leads to the up-regulation of those mediators.

Findings of the interplay between keratinocytes and peripheral sensory neurons (PSN) may aid to treat and/or prevent a variety of dermatological conditions and disorders, for instance, the wound healing and skin aging. During skin aging, a decrease in neurotrophicity, proliferation, differentiation, and number and rate of neuritis result in reduced skin sensations and skin thickness. Thus, a restoration of sensitive free nerve endings should re-establish the skin neurosensation and the epidermal thickness with an increased trophicity toward the epidermis, increase of neuritogenesis and, consequently, the skin innervation.

In accordance with the aforementioned, the aim of the present invention is to provide a neuroskin in the form of a kit comprising a co-culture microdevice containing peripheral sensory neurons (PSN) and human epidermal keratinocytes (HEK) in a suitable cell culture medium that allow assessing the interplay between sensory free endings and epidermal keratinocytes. Such kit is intended to study the biology and pharmacology of sensory free endings and epidermal keratinocytes interplay and screen potential new drugs of interest for cosmetic industry.

### SUMMARY OF THE INVENTION

The present invention discloses a new and effective kit comprising a co-culture microdevice, peripheral sensory neurons (PSN), human epidermal keratinocytes and a suitable cell culture medium that mimic the connection between free nerve endings and epidermal keratinocytes in the human skin.

Such neuroskin is advantageously used in a method for screening an active compound in vitro comprising the steps of (a) providing the kit according to the present invention and a test compound and (b) contacting said test compound with the kit and measuring the peripheral sensory function, wherein measuring the function consists of measuring the activity of at least one neuronal marker.

In addition, the present invention also discloses the use of the kit defined herein for performing in vitro tests and for producing a cosmetic product.

### BRIEF DESCRIPTION OF THE DRAWINGS

FIG. 1 shows the immunohistochemistry for (A) Nestin, (B) β-Tubulin III (TUJ1), (C) Peripherin, (D) TRPV1, (E) Nav1 and (F) CGRP in neural crest progenitor cells (NCPC) obtained from human induced pluripotent stem cells (hiPSC) and human neural stem cells (NSC). Cells are positive for all markers in both conditions.

FIG. 2 shows the quantification of neuronal markers (A) Nestin, (B)-Tubulin III (TUJ1), (C) Peripherin, (D) TRPV1, (E) Nav1 and (F) CGRP expressed by neural crest progenitor cells (NCPC) obtained from human induced pluripotent stem cells (hiPSC) and human neural stem cells (NSC).

FIG. 3 shows the neural cells 5 days after plating in the co-culture microdevice cultured with 20 μg/mL laminin (A) and 5 μg/mL laminin (B).

FIG. 4 shows neuronal aggregates in lower (A) and higher (B) co-culture microdevices.

FIG. 5 shows photomicrographs of peripheral sensory neurons (PSN) displaying healthy axons and the presence of growth cones.

FIG. 6 shows immunohistochemistry for (A) β-Tubulin III (TUJ1), (B) Peripherin, (C) Nuclei stained with DAPI and (D) merge.

FIG. 7 shows peripheral sensory neurons (PSN) co-cultured with keratinocytes in the co-culture microdevice. Neurites migrate through the canaliculi to the keratinocyte chamber. Red arrows indicate varicosities.

FIG. 8 shows photomicrographs displaying the cellular heterogeneity observed after the differentiation protocol. F: fibroblast-like, P: pyramidal neuron-like,?: unidentified morphology.

FIG. 9 shows neurospheres obtained from neural crest progenitor cells (NCPC).

FIG. 10 shows neurospheres plated on the co-culture device, displaying reduced cellular heterogeneity.

FIG. 11 shows the tropism of neurites from neurospheres towards human epidermal keratinocytes (HEK). A: co-culture microdevice. B: neurites migrating from the neurospheres towards human epidermal keratinocytes (HEK) chamber. C: contact between neurites and human epidermal keratinocytes (HEK). D: presence of varicosities.

FIG. 12 shows the immunohistochemistry for β-Tubulin III (TUJ1, green), Peripherin (red), Nuclei were stained with DAPI.

FIG. 13 shows the co-culture microdevice with added middle punch hole. Red arrows in A indicates the punched hole. Blue arrow shows line drawn to measure neurite growth.

FIG. 14 shows the quantification of neurite growth depending on culture conditions in the co-culture microdevice.

FIG. 15 shows that matrigel prevents the migration of human epidermal keratinocytes (HEK). A: HEK cells cultivated over matrigel are restricted to the hole area (red arrow), B: HEK cells cultivated without matrigel can be seen invading the canaliculi of the co-culture microdevice (blue arrow).

### DEFINITIONS

Stem cells are undifferentiated cells defined by their ability, at the single cell level, to both self-renew and differentiate. Stem cells may produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm, and ectoderm). Stem cells also give rise to tissues of multiple germ layers following transplantation and contribute substantially to most, if not all, tissues following injection into blastocysts. Stem cells are classified by their developmental potential.

As used herein, the term “pluripotent” refers to an ability to develop into the three developmental germ layers of the organism including endoderm, mesoderm, and ectoderm.

Characteristics of pluripotent stem cells are well known to those skilled in the art, and additional characteristics of pluripotent stem cells continue to be identified. Pluripotent stem cell markers include, for instance, the expression of one or more of the following: ABCG2, cripto, FOXD3, CONNEXIN43, CONNEXIN45, OCT4, SOX2, NANOG, hTERT, UTF1, ZFP42, SSEA-3, SSEA-4, TRA-1-60, TRA-1-81. In one embodiment, the pluripotent stem cells suitable for use in the present invention express one or more of NANOG, SOX2, TRA-1-60 and TRA-1-81, and lack expression of a marker for differentiation neural markers Islet1, BRN3A, peripherin and TRPV1.

The term “human induced pluripotent stem cells (hiPSC)”, as used herein, refers to a stem cell induced from a somatic cell, e.g., a differentiated somatic cell, and that has a higher potency than said somatic cell. Human induced pluripotent stem cells (hiPSC) are capable of self-renewal and differentiation into mature cells, such as neural crest progenitor cells (NCPC).

The term “human peripheral sensory neurons (PSN)”, as used herein, refers to main neuronal types present on skin layers, such as dermis and epidermis, interacting with skin cells and structures, such as epidermal keratinocytes, fibroblasts, melanocytes, sweat glands, hair follicles, etc.

“Cell culture” or “culturing” generally refer to cells taken from a living organism and grown under controlled conditions (“in culture” or “cultured”). A primary cell culture is a culture of cells, tissues, or organs taken directly from an organism before the first subculture. Cells are expanded in culture when they are placed in a growth medium under conditions that facilitate one or both of cell growth and division, resulting in a larger population of the cells. When cells are expanded in culture, the rate of cell proliferation is sometimes measured by the amount of time needed for the cells to double in number (referred to as doubling time).

A culture vessel used for culturing the stem cell(s) can include, but is particularly not limited to: flask, flask for tissue culture, dish, petri dish, dish for tissue culture, multi dish, micro culture vessel, micro-well plate, multi plate, multi-well plate, micro slide, chamber slide, schale, tube, tray, culture bag, and roller bottle, as long as it is capable of culturing the stem cells therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used).

Culturing conditions can be appropriately defined. For example, the culturing temperature can be about 30 to 40° C. and preferably about 37° C. but particularly not limited to it. The CO₂ concentration can be about 1 to 10% and preferably about 2 to 5%. The oxygen tension can be 1-10%.

Differentiation is the process by which an unspecialized (“uncommitted”) or less specialized cell acquires the features of a specialized cell such as, a nerve cell or a muscle cell. A differentiated cell or a differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the term “inhibitor” refers to a compound that reduces or abolishes the biological function or activity of the recited signaling pathway, by interfering with a specific target that is part of this signaling pathway or by interfering with the interaction between two or more targets. An inhibitor may perform any one or more of the following effects in order to reduce or abolish the biological function or activity of the protein to be inhibited: (i) the transcription of the gene encoding the protein to be inhibited is lowered, i.e. the level of mRNA is lowered, (ii) the translation of the mRNA encoding the protein to be inhibited is lowered, (iii) the protein performs its biochemical function with lowered efficiency in the presence of the inhibitor, and (iv) the protein performs its cellular function with lowered efficiency in the presence of the inhibitor.

Such compounds may include, without being limited to, small molecule, peptide, peptidomimetic, natural compound, siRNA, anti-sense nucleic acid, aptamer, or antibody.

In other words, an inhibitor is any compound or molecule that changes any activity of a named protein (signaling molecule, any molecule involved with the named signaling molecule, a named associated molecule, such as a glycogen synthase kinase 3β (GSK3β), for instance, via directly contacting SMAD signaling, contacting SMAD mRNA, causing conformational changes of SMAD, decreasing SMAD protein levels, or interfering with SMAD interactions with signaling partners, and affecting the expression of SMAD target genes.

Inhibitors also include molecules that indirectly regulate SMAD biological activity by intercepting upstream signaling molecules (e.g., within the extracellular domain, examples of a signaling molecule and an effect include: Noggin which sequesters bone morphogenic proteins, inhibiting activation of ALK receptors 1, 2, 3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules. Antibodies that block activins, nodal, TGFβ, and BMPs are contemplated for use to neutralize extracellular activators of SMAD signaling, and the like).

The term “mitogens”, as used herein, refers to those compounds that are members of the family of fibroblast growth factors, such as FGF-2 (basic FGF), and FGF-4. Also exemplary is epidermal growth factor (EGF), functional homologs, and other factors that bind the EGF receptor. Other candidate growth factors are platelet-derived growth factor (PDGF), insulin-like growth factor (IGF). These mitogens are used for increasing the number of a lineage cells, causing them to proliferate further in a culture.

“Neurotrophic factors” are endogenous peptides, found in the nervous system or in non-nerve tissues innervated by the nervous system, that function to promote the survival and maintain the phenotypic differentiation of nerve and/or glial cells. The family of trophic factors, called the neurotrophins, currently includes brain-derived neurotrophic factor (BDNF), nerve growth factor (NGF), glial cell line-derived neurotrophic factor (GDNF), neurotrophin-3 (NT-3), NT-4/5, and NT-6. All neurotrophic factors can be used isolated or in combination.

Preferred amounts of each neurotrophic factor to be employed is between about 1 ng/ml and about 25 ng/ml, more preferably between about 5 ng/ml and about 15 ng/mL, more preferably about 10 ng/ml.

As used herein, the expression “differentiation inductor”, refers to the ascorbic acid (AA).

Preferred amounts of the differentiation inductor to be employed is between about 50 μM and about 500 μM, more preferably between about 100 μM and about 300 μM, more preferably about 200 μM.

As used herein, the expression “cell transduction inductor” refers to a compound, which mediates signal transduction, such as, for example CAMP.

Preferred amounts of the cell transduction inductor to be employed is between about 0.01 mM and about 1 mM, more preferably between about 0.1 mM and about 0.8 mM, more preferably about 0.5 mM.

“Markers”, as used herein, are nucleic acid or polypeptide molecules that are differentially expressed in a cell of interest. In this context, differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive for” a specific marker or “positive” when the specific marker is sufficiently detected in the cell. Similarly, the cell is “negative for” a specific marker, or “negative” when the specific marker is not sufficiently detected in the cell.

As used herein, the term “activator” “activating” refers to compounds for activating molecules resulting in directed differentiation of cells of the present invention. Exemplary activators include but are not limited to: noxious heat/cold, mechanical stimulation, chemical stimuli (menthol, piperine, acute capsaicin, cinnamaldehyde, resiniferatoxin, bradykinin, ATP, prostaglandins, inflammatory cytokines, acidic saline, fibroblast growth factor (FGF), etc).

Active compounds, as referred herein, refer to known cosmetic ingredients, dermatological and biological actives, such as neuromodulators, anti-aging compounds, neuroaging regulators, to control free nerve endings growth rate and density, electrical conductance along skin layers, trigger action potential in peripheral sensory neurons, increase interaction between these neurons and other skin cell types as keratinocytes, fibroblasts and melanocytes, adipocytes, hair follicle cells, glands, cartilage, stem cells, etc.

As referred herein, TRP (transient receptor potential) channels comprehend a diverse family of ligand-gated, mostly non-selective, cation channels that are robustly expressed in sensory systems throughout species (Nilius & Szallasi, Transient receptor potential channels as drug targets from the science of basic research to the art of medicine. Pharmacol Rev 2014; 66 (3): 676-814). Of these, TRPV1 is the most well studied and considered the prototypical TRP channel present in somatosensory neurons (Basbaum et al., Cellular and molecular mechanisms of pain. Cell; 2009; 139:267-284). TRPV1 can be directly gated by external molecules such as capsaicin, resiniferatoxin and piperine, and also modulated positively or negatively via activation of other receptors and second messenger systems, such as PIP2 hydrolysis and PKC phosphorylation (Julius, TRP channels and pain. Annu Rev Cell Dev Biol. 2013; 29:355-84). One of the receptors that seem to inhibit TRPV1 activation is the cannabinoid 1 receptor (CB1), also present in somatosensory neurons (Julius & Basbaum, Molecular mechanisms of nociception. Nature. 2001; 413:203-210). However, an endogenous agonist of CB1, anandamide, is also an agonist of TRPV1, albeit with a EC50 an order of magnitude higher in the latter (Zygmunt et al., Vanilloid receptors on sensory nerves mediate the vasodilator action of anandamide. Nature. 1999; 400:452-457).

Substance P (SP) is a neuropeptide member of the tachykinin family, synthesized by sensory neurons that emit their extensions from the DRG to the more superficial layers of the skin, mediating the communication between peripheral neurons and epidermal keratinocytes (Ribeiro-da-Silva & Hokfelt, Neuroanatomical localization of Substance P in the CNS and sensory neurons. Neuropeptides. 2000; 34:256-271). Most of the neurons that release substance P are sensitive to capsaicin, highlighting the importance of TRPV1 expression and sensory neurons-keratinocytes interplay.

### DETAILED DESCRIPTION OF THE INVENTION

The present invention is directed to a novel and particular kit comprising:
(i) a co-culture microdevice;
(ii) peripheral sensory neurons (PSN);
(iii) human epidermal keratinocytes (HEK); and
(iv) a cell culture medium.

According to the present invention, the peripheral sensory neurons (PSN) are derived from human neural stem cells (NSC) or human induced pluripotent stem cells (hiPSC). Preferably, the peripheral sensory neurons (PSN) are derived from human induced pluripotent stem cells (hiPSC).

The hiPSC-derived peripheral sensory neurons (PSN) may be obtained by any method known in the art. In a preferred embodiment, the method from inducing differentiation to peripheral sensory neurons (PSN) comprises the steps of contacting human stem cells, such as human induced pluripotent stem cells (hiPSC) with at least one SMAD pathway inhibitor, in a neural induction medium, to produce primarily differentiated cells, such as neural crest progenitor cells (NCPC), and obtain therefrom human peripheral sensory neurons (PSN) by culturing the primarily differentiated cells with at least one mitogen, one neurotrophic factor, one differentiation inductor and one cell transduction inductor.

In a preferred embodiment, the peripheral sensory neurons (PSN) are induced to spontaneously form neurospheres during the maturation of the neurons in order to reduce the heterogeneity of the cultures.

The cell culture medium of the present invention is any suitable medium adapted for both cell types, i.e., for peripheral sensory neurons (PSN) and human epidermal keratinocytes. In a preferred embodiment, the cell culture medium is 3N medium, in particular, a 3N medium comprising a 1:1 mixture of N2-containing medium and B27-containing medium.

In a preferred embodiment, the N2-containing medium comprises DMEM/F12 supplemented with N2 supplement (GIBCO), insulin, L-glutamine, non-essential amino acids (NEAA), β-mercaptoethanol, penicillin and streptomycin. In a preferred embodiment, the B27-containing medium comprises neurobasal medium (Invitrogen) supplemented with B27 supplement (GIBCO), L-glutamine, penicillin and streptomycin.

Preferably, the 3N medium is supplemented with NGF, TGFβ and/or BMP signaling inhibitors to produce a neural induction medium.

In a preferred embodiment, the cell culture medium is provided with a medium gradient between the two cell types.

According to the present invention, the co-culture microdevice is a microfluidic device containing several microchannels, inside which the peripheral sensory neurons (PSN) grow orderly allowing a good connection with the human epidermal keratinocytes (HEK). Advantageously, the co-culture microdevice is used in the horizontal setup to provide improved culture conditions to connect the neurons with the keratinocytes.

In a preferred embodiment, the co-culture microdevice is a microchip for cell culture made of biocompatible silicone and comprising four to twenty independent chambers, wherein skin cell types, such as the peripheral sensory neurons (PSN) and human epidermal keratinocytes (HEK), are plated alternatively in each side of the independent chambers, having a channel diameter that allows the axonal elongation and neurite growth thought the interchamber microchannels in the chip, while preventing the cell body migration to the other chamber and the invasion of the co-cultured cell compartment space.

Preferably, the co-culture microdevice comprises a hole wherein the human epidermal keratinocytes (HEK) are plated.

Still in a preferred embodiment, the co-culture microdevice is covered with matrigel or laminin or poly-ornithine or collagen to create a proper microenvironment for each cell type and to prevent the migration of the human cells to other compartment, as well as to allow the neuronal growth giving mechanical cues inducing neuronal fate of the neural crest progenitor cells (NCPC) or neuronal progenitor cells (NPC) and epidermal keratinocytes (HEK) through the device chambers.

Another object of the present invention relates to a method for screening an active compound in vitro comprising:
(a) the provision of:
(i) a kit as defined herein;
(ii) a test compound; and
(b) contacting said test compound with the kit and measuring the peripheral sensory neuron function, wherein measuring the function consists of measuring the activity of at least one neuronal marker.

Preferably, the active compound screened by the method of the present invention acts, for instance, on the modulation of neuronal growth, number of nerve endings, neuronal activity and epidermal regeneration. More preferably, the modulation of neuronal activity is mediated by the induction of growth factor release by human epidermal keratinocytes (HEK) and the epidermal regeneration is mediated by the modulation of neuronal release of factors.

In a preferred embodiment, the active compound screened by the method of the present invention is for treating and/or preventing, for instance, atopic dermatitis, sensitive skin, photoaging and photopollution impacts, neuroaging, wound healing, neuron-controlled skin barrier function, itching, skin mechano-sensoriality and epidermal thickness in aged skin.

A further object of the present invention is the use of the kit as defined herein for performing in vitro tests, preferably, tests for screening an active compound that acts, for instance, on the modulation of neuronal growth, number of nerve endings, neuronal activity and epidermal regeneration. More preferably, the modulation of neuronal activity is mediated by the induction of growth factor release by human epidermal keratinocytes (HEK) and the epidermal regeneration is mediated by the modulation of neuronal release of factors.

In a preferred embodiment, the use of the kit of the present invention is to screen an active compound for treating and/or preventing, for instance, atopic dermatitis, sensitive skin, photoaging and photopollution impacts, neuroaging, wound healing, neuron-controlled skin barrier function, itching, skin mechano-sensoriality and epidermal thickness in aged skin.

It was surprisingly and unexpectedly discovered that the novel and particular kit comprising a co-culture microdevice, peripheral sensory neurons (PSN), human epidermal keratinocytes (HEK), and a cell culture medium of the present invention allow the peripheral sensory neurons (PSN) to grow orderly and connect with the human epidermal keratinocytes (HEK), so that mimicking the connection between sensory free nerve endings and epidermal keratinocytes (HEK) in human skin.

The following examples are intended to illustrate the invention with reference to some preferred embodiments, without being limited to the details shown. Rather, various modifications may be made in the details without departing from the scope of the invention.

### EXAMPLES

### Example 1

### Method from Inducing Differentiation to Peripheral Sensory Neurons (PSN)

Differentiation of Human Induced Pluripotent Stem Cells (hiPSC) to Neural Crest Progenitor Cells (NCPC)

Human induced pluripotent stem cells (hiPSC) were cultured in Essential 8 medium (Thermo Fisher Scientific, USA) on matrigel (BD Biosciences)-coated dishes in standard culture conditions (37° C., 5% CO₂). The colonies were split using 0.5 mM EDTA (Thermo Fisher Scientific, USA) every 4-5 days. Human iPS cell cultures at 40-70% confluence were used for NCPC induction. hiPSCs were exposed for 10 days to chemically defined 3N induction medium (DMEM+Neurobasal medium 50:50 v/v, 1% Glutamax, 0.5% N2, 1% B27, 0.5% NEAA, 55 mM β-mercaptoethanol and 1% Penicillin/Streptomycin, all from Thermo Fisher Scientific, USA) freshly supplemented with three small-molecule compounds. The addition of these compounds was as following: day 1:500 nM LDN (Stemgen, USA)+10 μM SB (Sigma Aldrich, USA); day 2:500 nM LDN+10 μM SB+3 μM CHIR (Tocris Bioscience, USA); day 3:10 μM SB+3 M CHIR. At days 4, 6 and 8, the medium was supplemented only with 3 μM CHIR. After 10 days of differentiation, NCPCs were further cultured in expansion medium (3N medium freshly supplemented with 10 ng/ml BFGF and 10 ng/ml EGF, both from Thermo Fisher Scientific, USA). At day 11, NCPCs were enzymatically passaged (passage 0) using Accutase (Merck Millipore, USA) for 2-3 min at 37° C. and split 1:3 onto Poly-L-ornithine (100 μg/mL, Sigma Aldrich, USA)/Laminine (20 μg/mL, Thermo Fisher Scientific, USA)-coated dishes and cultured until confluent. The medium was replaced every other day. When 70-100% confluence was reached (normally 24-48 h after passage 0), the cells were passaged again and cultured in a culture vessel at specific densities: 1×10⁶ cell per 60 mm dish or 3×10⁶ cell per 100 mm dish. 10 μM ROCK inhibitor (Merck Millipore, USA) was added at the day of passaging and removed 24 h after.

Generation of Peripheral Sensory Neurons (PSN) from NCPCs

Neural crest progenitor cell (NCPC) cultures at approximately 80% confluence (usually at day 13) were used for neuronal differentiation. Briefly, the cells were maintained for approximately 23 days in neural induction medium containing the following differentiation factors: 0.5 mM AMPc (Sigma Aldrich, USA), 200 μM AA (Sigma Aldrich, USA), 10 ng/mL NT-3 (R&D Systems, USA), 10 ng/ml NGF (R&D Systems, USA), 10 ng/ml BDNF (R&D Systems, USA) and 10 ng/ml GDNF (R&D Systems, USA). The medium was replaced every 3-4 days. The neurons were enzymatically split (if necessary) using Accutase (Merck Millipore, USA) for 3-5 min at 37° C. onto freshly prepared Poly-Lornithine/Laminine dishes. The addition of 10 μM ROCK inhibitor (Merck Millipore, USA) was applied at every passage to increase the survival and attachment ability of the neurons. At day 35, approximately, they neurons were harvested and cultured in a culture vessel for analysis and/or further experiments.

### Human Epidermal Keratinocytes (HEK) Culture

Neonatal human epidermal keratinocytes (HEKn) were obtained from Cascade Biologics (Portland, OR) and cultured in EpiLife serum-free medium (ThermoFischer). Cells were cultured in a culture vessel at 10,000 cell per well. Culture vessels previously treated with gelatin (Sigma) and medium EpiLife (Thermo Fisher Scientific) split when 70% to 75% confluence 48 h conditioning and then it was collected added fresh to the neurons medium was centrifuged to get rid of debris and dead cells.

Co-Culture of Human PSN and HEK and Treatment with Conditioned Media

On day 35 of neural differentiation, peripheral sensory neurons were harvested and cultured in a culture vessel at 30,000 cell per well onto 96-well (Perkin-Elmer, USA) Poly-L-ornithine/Laminine-coated culture vessels for additional two, five and ten days under following conditions: in co-culture with HEKn cells in standard neural induction medium; and without HEKn cells but with addition of HEKn-conditioned medium at three different proportions (25, 50 and 75%). Conditioned media were exchanged every 3 days.

### Example 2

### Comparison Between NCPCs Obtained from iPS and Neural Stem Cells (NSC)

### Immunocytochemistry

Neural crest progenitor cells (NCPC) obtained from hiPSC and NSC were cultured in 96-well culture vessels and fixed with 4% paraformaldehyde, permeabilized with Triton X-100 and blocked with 3% bovine serum albumin (BSA). Cells were incubated for 2 hours with primary antibodies diluted in 3% BSA. After washing with PBS, conjugated secondary antibodies were added for 40 minutes in the dark, washed thoroughly with PBS followed by a 5-minute incubation with DAPI (4′,6-diamidino-2-phenylindole) for nuclear staining. After rinsing with PBS and water, 50 μl of glycerol was added as mounting media and the culture vessels were sealed with aluminum sticker before analysis. The primary antibodies used were: Nestin (1:100, Sigma-Aldrich, USA), anti-β-tubulin III (1:200, Merck-Millipore, Germany), anti-peripherin (1:250, Santa Cruz Biotechnology), anti-TRPV1 (1:1000, Abcam), anti-Nav1 (1:1000, Abcam), anti-CGRP (1:250, Santa Cruz Biotechnology). Secondary antibodies conjugated with Alexa Fluor 488 and Alexa Fluor 594 (1:400, Life Technologies, USA) were incubated for 40 minutes protected from light. Nuclei were stained with 0.5 μg/mL 4′-6-diamino-2-phenylindole (DAPI) for 5 min. Images were acquired using a High-Content Screening microscope, Operetta (PerkinElmer, USA) and analysis were performed using high-content image analysis software Harmony 5.1 (PerkinElmer, USA).

### Tests

Neural stem cells (NSCs) are produced from iPS cells and can be differentiated into neurons and glial cells. They are easy to handle and can go through several freeze/thaw cycles without losing the ability to differentiate. Obtaining NCPCs from NSCs could avoid possible loss of efficiency in the differentiation of post-thaw NCPCs.

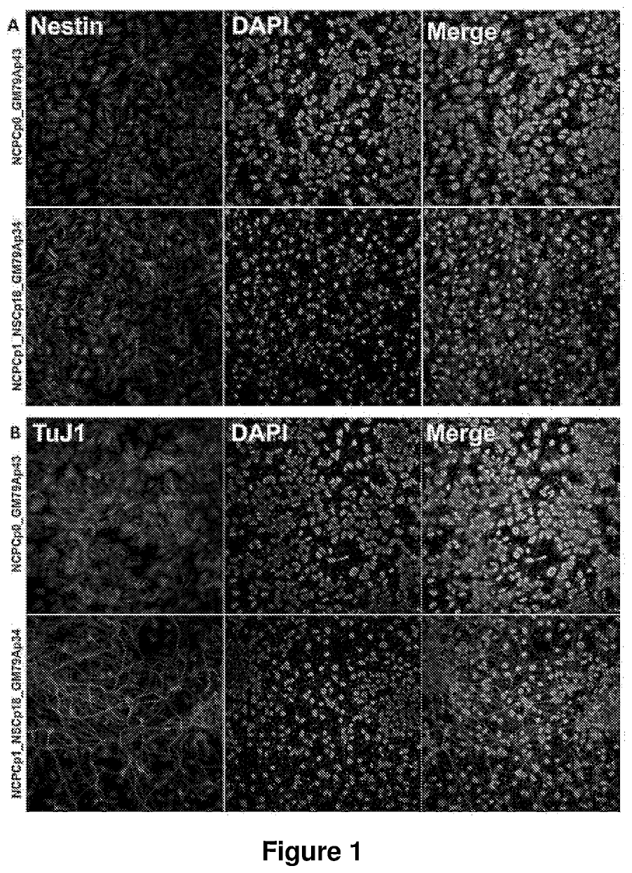

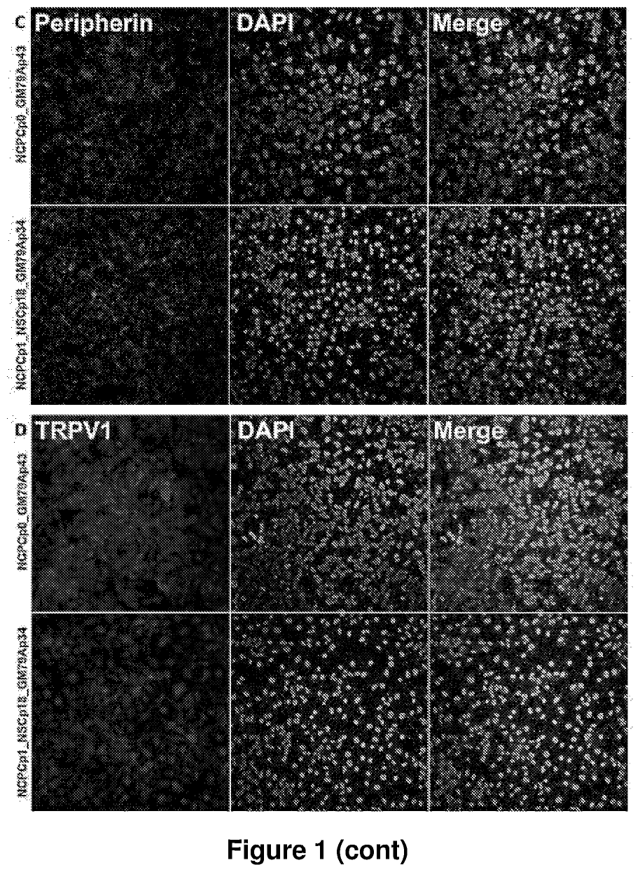

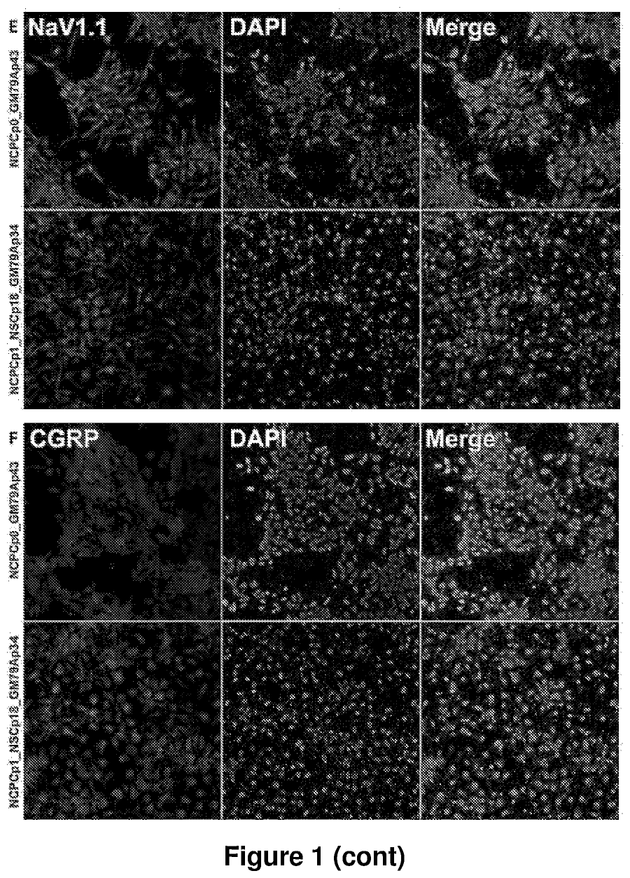

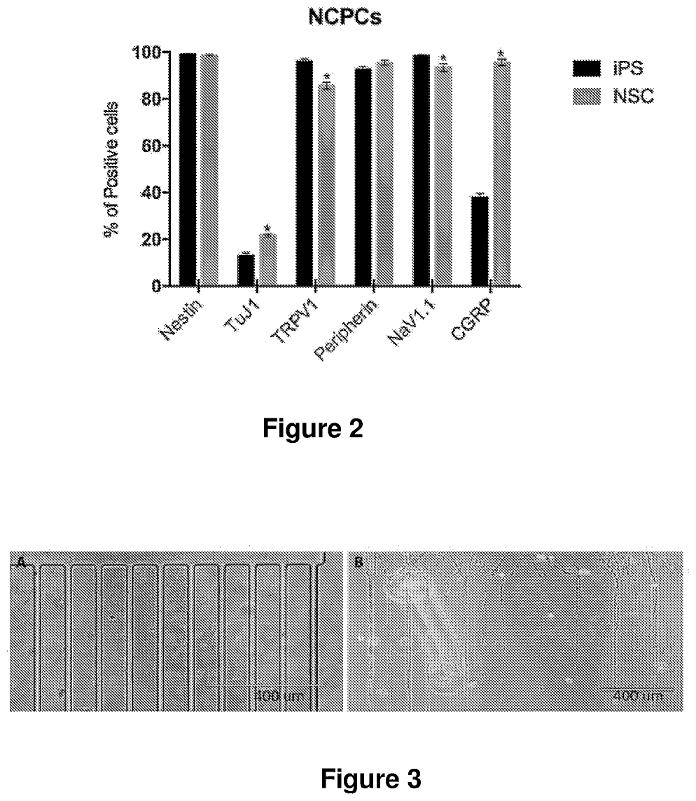

The expression of the neural markers: Nestin, β-Tubulin III (TUJ1), Peripherin, TRPV1, Nav1 and CGRP in NCPCs derived from iPS and from NSC were compared. At first, the results suggested that both present similar expression patterns confirming their neural stem cell fate (FIG. 1). However, some differences when the immunolabelings were quantified were observed (FIG. 2).

### Example 3

### Strategies to Increase PSN Maturity In Vitro and in Co-Culture

Some co-culture microdevices were tested and various parameters described hereinafter were adjusted in order to find the best configuration for PSN growth and subsequent co-culture with HEK.

Neuronal differentiation requires the use of extracellular matrices capable of allowing its development and migration, like laminin. 20 μg/mL laminin are typically used in conventional protocols. This concentration, however, caused clogging of the canaliculi of the microdevices and disorganization in the growth of neurites (FIG. 3A). After adjusting the laminin concentration to 5 μg/mL, neurites grew 11 μm in 5 days, following a straight path (FIG. 3B).

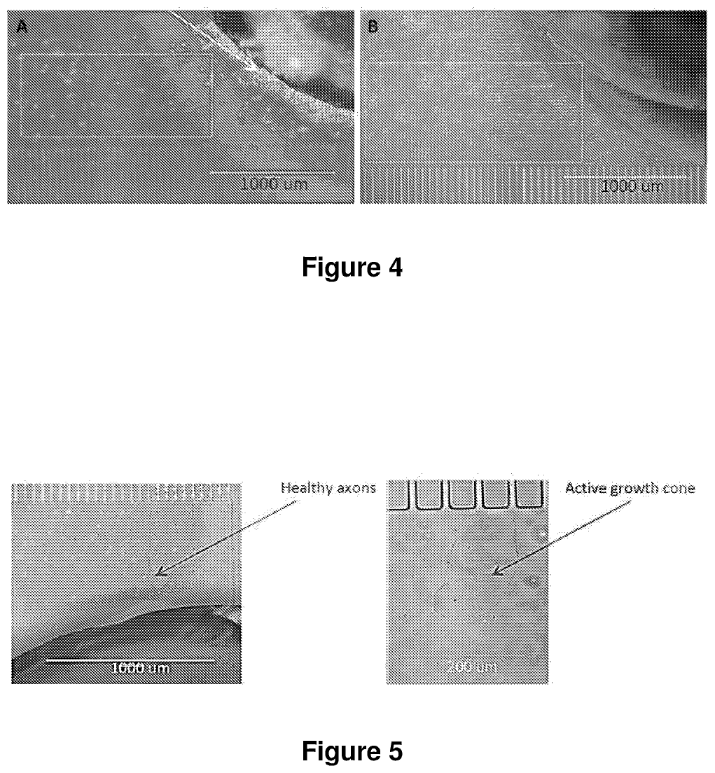

The second step was to adapt the microdevice to the particular cellular model of the present invention. The differentiation protocol promotes the formation of aggregates from which neurons migrate. The first microdevices did not allow the entrance of aggregates, reducing the final number of neurons (FIG. 4A). The height change allowed the entry of aggregates and the uniform distribution of neurons within the microdevice (FIG. 4B).

Subsequently, it was performed the co-cultivation of PSN with keratinocytes. These cells, however, did not adapt well to the neuronal medium. So, PSNs were cultivated with keratinocyte-conditioned medium, which promoted an increase in the number of neuronal processes and more abundant expression of markers such as TRPV1.

In such test, different proportions of neuronal medium are mixed with keratinocyte medium. The combination consisting of 75% keratinocyte medium and 25% neuronal medium seemed to be sufficient to keep the neurons viable and healthy (FIG. 5).

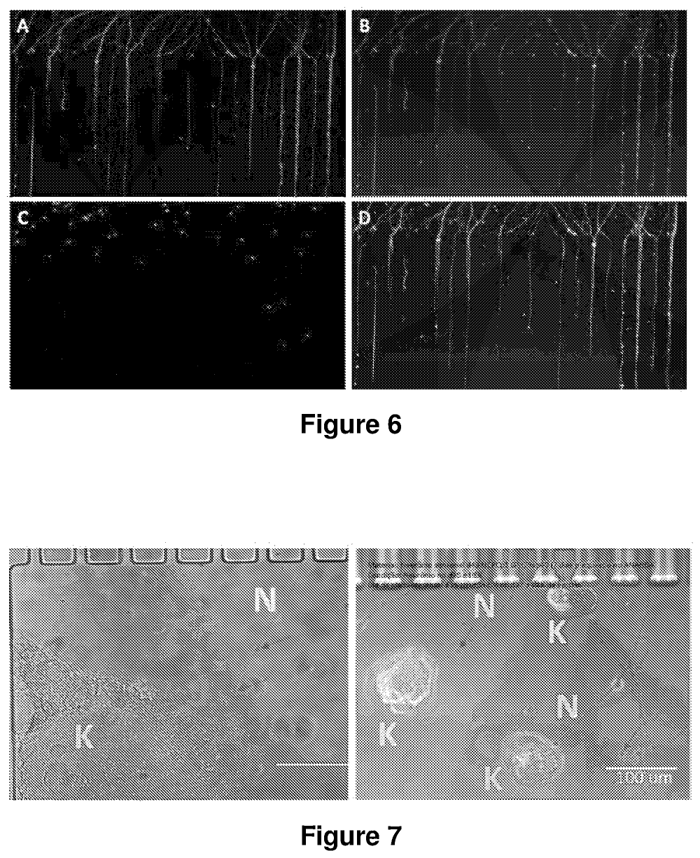

When cultured under these conditions, neurons are also positive for β-Tubulin III (TUJ1) and peripherin (FIG. 6).

After this, it was tested plating each cell type at different times (e.g., PSNs and then keratinocytes and vice-versa), as well as modifications in the flow between chambers. It was observed that neurons emitted their processes to the keratinocyte chamber, suggesting interaction between cells (FIG. 7). It is interesting to note that neurons presented varicosities (red arrows), a characteristic found in skin biopsies (Cauna, 1980; Mccarthy, B. G. et al., 1995; Talagas et al., 2018). It results from the intense transport of vesicles, performed particularly by these cells.

### Example 4

### Differentiation-Induced Variability

Recently, Schwartzentruber et al. (2017) carried out a large-scale study with 123 differentiation procedures of sensory neurons from iPS cells. They observed cellular heterogeneity on each differentiation. Indeed, iPS cells showed a greater degree of variability than embryonic stem cells, although they were able of generating the same cell types within the same time (Hu B Y et al., 2010).

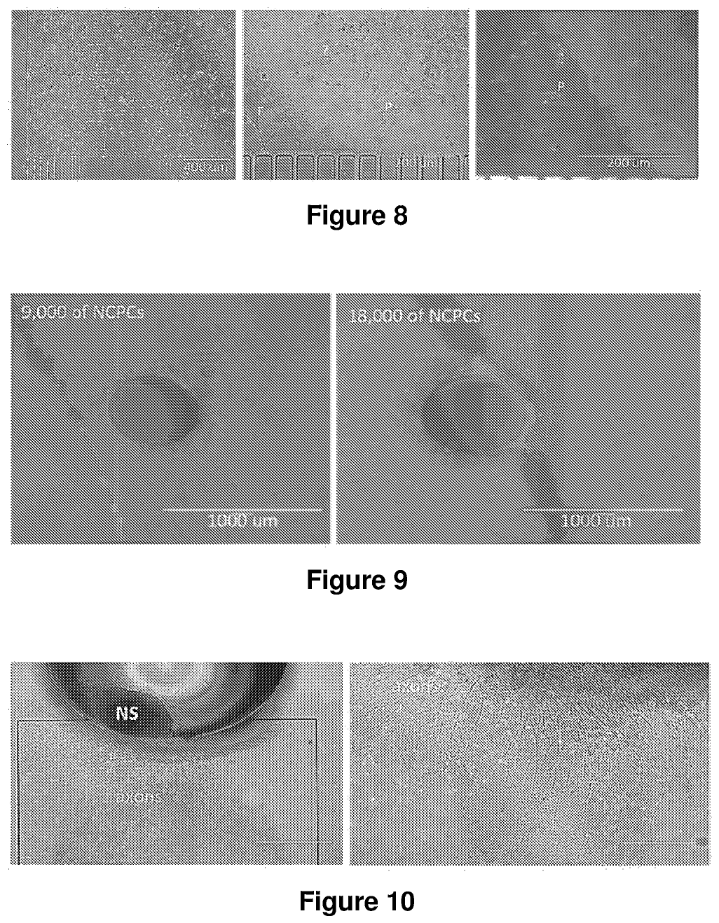

In the protocol of the present invention, variability in the morphology of cells varying in proportion to each differentiation were also observed (FIG. 8).

In an attempt to reduce heterogeneity of cultures, strategies used in other protocols for neuronal differentiation were adopted. After the production of NCPCs, the formation of neurospheres was induced, such neurospheres were spontaneously formed as the neurons matured, albeit the heterogeneity. Therefore, it was concluded that by controlling the neurosphere formation, the heterogeneity may be reduced, potentially facilitating differentiation. Cell density is a crucial factor for the correct formation of neurospheres. So, two amounts of cells, 9×10³ and 18×10³, were used and it was discovered that the smallest amount formed the healthier and more homogeneous neurospheres, with very similar yield (FIG. 9).

### Example 5

### Differentiated Neurospheres in Microfluidic Devices

After successfully differentiating neurospheres as described, they were plated and, as expected, there was a reduction in the number of contaminating cells and an exuberant migration of neurites (FIG. 10). In addition, there was a 2-week reduction in the differentiation time.

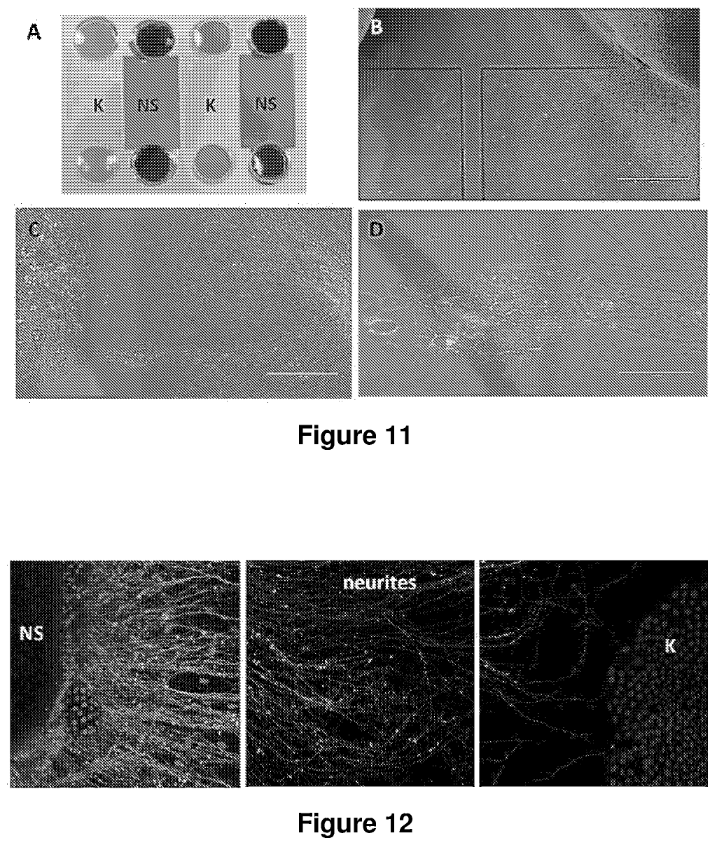

After that, co-culture microdevice having 4 independent chambers were used. Neurospheres (NS) and keratinocytes (K) were plated alternately (FIG. 11A). It was observed that neurites from neurospheres moved towards the keratinocyte chamber (FIG. 11B). After removal of the microdevice, contact regions between neurites and keratinocytes and the existence of varicosities suggesting interaction between cells were observed (FIGS. 11C-D).

Neurons obtained from NCPCs neurospheres are peripherin-positive, have varicosities and tropism for keratinocytes (FIG. 12).

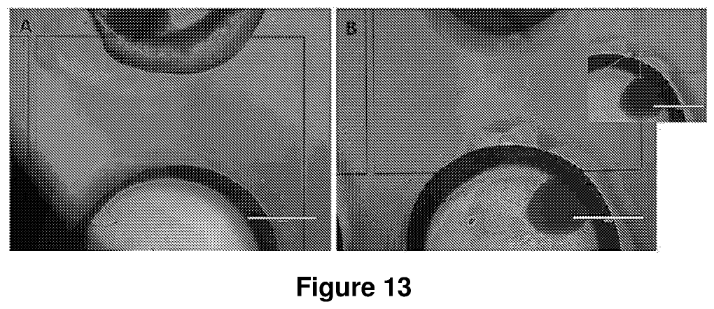

Keratinocytes moved faster than the dendrites and since removing the microdevice sometimes meant that neurospheres were pulled from the substrate, a hole between the wells of the same pair was punched and the keratinocytes were placed within this hole (FIG. 13A, red arrow). The region where there was a predominance of migrating neurites was observed and the highest migration radius was measured (FIG. 13B, blue arrow).

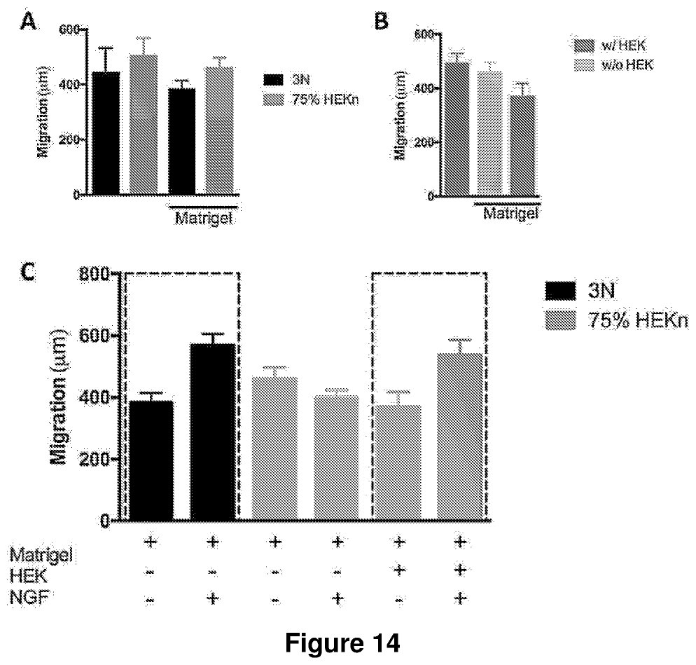

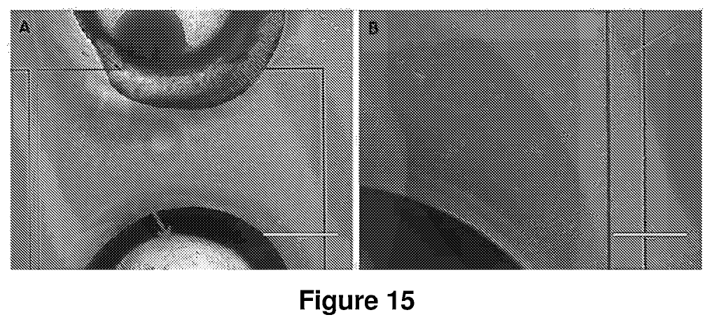

To further test this modified co-culture microdevice, the culture media (3N versus 75% CM from HEKn) and the presence (or not) of matrigel in the middle hole were compared by measuring the maximum extension of the neurites emerging from the neurospheres (FIG. 14A). There were no statistically significant differences. Then, the extension of neurites in the presence or absence of keratinocytes in the hole were compared. Again, no differences were observed (FIG. 14B). Finally, another variable was tested: adding NGF or not to the matrigel in the middle punched hole. Although no statistical significance was achieved when comparing treatments, it was evident the trend of NGF to increase migration, particularly between in 3N medium (first and second bars in the graph) and the groups with 75% CM plus HEKn (5th and last bars) (FIG. 14C).

It was also noticed that matrigel prevented HEK from coming out of the hole (FIG. 15A). In the absence of matrigel, HEK tends to migrate through the device (FIG. 15B).

1-26. (canceled)

27. A method for screening activity of a compound, the method comprising:
(a) contacting the compound with a co-culture microdevice comprising at least two compartments, wherein:
(i) a first compartment comprises peripheral sensory neurons,
(ii) a second compartment comprises human epidermal keratinocytes, and
(iii) the co-culture microdevice comprises a cell culture medium with a medium gradient between the two compartments,
(b) measuring the peripheral sensory neuron function.

28. The method according to claim 27, wherein the peripheral sensory neurons are cells that differentiated from human induced pluripotent stem cells.

29. The method according to claim 27, wherein the cell culture medium comprises a mixture of N−2-containing medium and B-27-containing medium.

30. The method according to claim 29, wherein the N−2-containing medium comprises one or more of DMEM/F12 supplemented with N−2 supplement, insulin, L-glutamine, non-essential amino acids, β-mercaptoethanol, penicillin, and streptomycin.

31. The method according to claim 29, wherein the B-27-containing medium comprises one or more of neurobasal medium supplemented with B-27 supplement, L-glutamine, penicillin, and streptomycin.

32. The method according to claim 29, wherein the cell culture medium further comprises TGFβ and/or BMP signaling inhibitors sufficient to produce a neural induction medium.

33. The method according to claim 27, wherein the co-culture microdevice is a microfluidic device containing microchannels.

34. The method according to claim 27, wherein the co-culture microdevice is a microchip for cell culture made of biocompatible silicone and comprising from four to twenty independent chambers, wherein the peripheral sensory neurons and human epidermal keratinocytes are plated alternatively in each side of the independent chambers.

35. The method according to claim 27, wherein the co-culture microdevice comprises a hole wherein the human epidermal keratinocytes are plated.

36. The method according to claim 27, wherein the co-culture microdevice is covered with a membrane comprising one or more of Matrigel, laminin, poly-ornithine, and collagen.

37. The method according to claim 27, wherein measuring the peripheral sensory neuron function comprises measuring the activity of at least one neuronal marker.

38. The method according to claim 27, wherein the compound is chosen from compounds that act on the modulation of neuronal growth, modulation of neuronal activity, number of nerve endings, and/or epidermal regeneration.

39. The method according to claim 27, wherein the compound is chosen from compounds that act on the modulation of neuronal activity mediated by the induction of growth factor release by human epidermal keratinocytes.

40. The method according to claim 27, wherein the compound is chosen from compounds that act on epidermal regeneration mediated by the modulation of neuronal release of factors.

41. The method according to claim 27, wherein the compound is an anti-aging compound.

42. The method according to claim 27, wherein the compound is chosen from compounds for treating and/or preventing atopic dermatitis, sensitive skin, photoaging, neuroaging, wound healing, neuron-controlled skin barrier function, itching, skin mechano-sensoriality, and/or epidermal thickness.

43. A method for screening activity of a compound, the method comprising:
(a) contacting the compound with a co-culture microchip comprising at least two chambers, wherein:
(i) a first chamber comprises peripheral sensory neurons,
(ii) a second chamber comprises human epidermal keratinocytes, and
(iii) the co-culture microchip comprises a cell culture medium with a medium gradient between the two chambers,
(b) measuring the peripheral sensory neuron function.

44. The method according to claim 43, wherein the compound is an anti-aging compound.

45. The method according to claim 43, wherein the compound is chosen from compounds for treating and/or preventing atopic dermatitis, sensitive skin, photoaging, neuroaging, wound healing, neuron-controlled skin barrier function, itching, skin mechano-sensoriality, and/or epidermal thickness.

46. The method according to claim 43, wherein measuring the peripheral sensory neuron function comprises measuring the activity of at least one neuronal marker.
